(19) United States
(12) Patent Application Publication
(10) Pub. No.: US 2016/0287380 A1
(43) Pub. Date: Oct. 6, 2016

(54) FLUID-FILLED IMPLANTABLE STRUCTURES WITH INTERNAL SURFACE-MODIFYING COMPONENTS AND RELATED METHODS

(71) Applicants: Wendian SHI, Monrovia, CA (US); Charles DEBOER, Sierra Madre, CA (US); Yu-Chong TAI, Pasadena, CA (US); Jonathan K. LEE, Montebello, CA (US); Ramiro Magalhaes RIBEIRO, South Pasadena, CA (US); Mark S. HUMAYUN, Glendale, CA (US)

(72) Inventors: Wendian SHI, Monrovia, CA (US); Charles DEBOER, Sierra Madre, CA (US); Yu-Chong TAI, Pasadena, CA (US); Jonathan K. LEE, Montebello, CA (US); Ramiro Magalhaes RIBEIRO, South Pasadena, CA (US); Mark S. HUMAYUN, Glendale, CA (US)

(21) Appl. No.: 15/085,324

(22) Filed: Mar. 30, 2016

Related U.S. Application Data
(60) Provisional application No. 62/139,937, filed on Mar. 30, 2015.
(60) Provisional application No. 62/143,274, filed on Apr. 6, 2015.

(51) Int. Cl. A61F 2/16

(52) U.S. Cl. CPC A61F 2/1601; A61F 2230/0071; A61F 2240/001

(57) ABSTRACT

Implantable reservoir structures include an interior and/or exterior modifying element joined to the interior and/or exterior surface of the membrane defining the reservoir in order to alter one or more physical properties thereof. The physical properties can be mechanical (e.g., material strength, flexibility, shear modulus, Young's modulus, hardness, and/or ductility); optical (e.g., refraction, transparency, transmission spectrum, absorption spectrum, fluorescence spectrum, and/or color); and/or permeability to liquids generally or to a particular type of liquid, solute, or suspended material.

### CROSS-REFERENCE TO RELATED APPLICATIONS

This application claims priority to and the benefit of U.S. Provisional Application No. 62/139,937, filed on Mar. 30, 2015, and U.S. Provisional Patent Application No. 62/143,274, filed on Apr. 6, 2015, the entire disclosure of each of which is hereby incorporated by reference.

### FIELD OF THE INVENTION

In various embodiments, the present invention relates generally to surgically implantable fluid-filled reservoirs and their manufacture.

### BACKGROUND

Fluid-filled implantable reservoirs (FFIRs) are used in numerous medical applications and in various anatomic sites. Such reservoirs may, for example, serve as intraocular lenses (IOLs), which are used to replace the natural crystalline lens after cataract removal; may be used to position tissue and/or provide tissue compression or spacing during surgery; and may deliver pharmaceuticals. In general, the envelope of the reservoir is unitary and its mechanical properties are therefore uniform. This is not always ideal. For example, during laser treatment for posterior capsule opacification (a thickening of the lens capsule of the eye that can result as a complication of cataract surgery), the posterior membrane of an IOL can be damaged while the anterior membrane is unaffected. For correction of some vision conditions, such as astigmatism, local variation in optical properties may be necessary. And in certain drug-delivery implants, it may be desirable to confine drug perfusion to a portion of the reservoir rather than permitting 360° radial broadcast.

Accordingly, there is a need for the ability to modify the mechanical, optical and/or permeability properties of a portion of the FFIR membrane with specificity and without effect on the remainder of the FFIR.

### SUMMARY

In various embodiments, the present invention facilitates local modification of only a portion of the envelope membrane of a FFIR. In particular, an interior and/or exterior modifying element may be fabricated on or joined to the interior and/or exterior surface of the membrane in order to alter one or more physical properties thereof. As used herein, the term “physical properties” broadly encompasses mechanical properties such as material strength (e.g., tensile or tear strength, yield strength, shear strength, compressive strength, flexural strength, specific strength, etc.), flexibility, shear modulus, Young's modulus, toughness, hardness (e.g., durometer), and ductility (i.e., ability to deform under a tensile load); optical properties such as refraction, transparency, transmission spectrum, absorption spectrum, fluorescence spectrum, and color; and permeability to liquids generally or to a particular type of liquid, solute, or suspended material. In other embodiments, the interior or exterior element may be a gel or other matrix containing a compound of interest, such as a pharmaceutical, that is released into the lumen of the FFIR for diffusion out of the FFIR and into the site of implantation. In still other embodiments, the interior or exterior element may contain a device such as a sensor.

When the FFIR is an intraocular lens (IOL), local modifications of specific portions of the envelope membrane of the FFIR may be particularly beneficial. Posterior capsule opacification (PCO) is a thickening of the lens capsule of the eye, and represents a fairly common complication of cataract surgery following implantation of an IOL (see, e.g., U.S. patent application Ser. No. 14/058,634, filed Oct. 21, 2013, the entire disclosure of which is incorporated by reference herein). The lens capsule is the thin, elastic, bag-like ocular structure that retains an IOL in place following implantation. During cataract surgery, the front (anterior) portion of the lens capsule is carefully opened and the cataract is removed. In the case of removal of the anterior portion or posterior portion of the capsule, it is beneficial for a FFIR to have comparably rigid anterior or posterior portions respectively to maintain a flexible yet conformal fit within the capsule while preventing excessive deformation of the FFIR at the opened portions.

Patients who experience PCO experience symptoms similar to those of the original cataract. Due to the high clearance rate of drugs from the vitreous, poor penetration of topical drops through the cornea, and systemic effects that can be caused by oral medication, PCO is difficult to prevent or treat using conventional drug delivery. While a fluid-filled IOL may be coated with a drug, this additional coating is of a short duration and may affect the optical properties and the accommodation of the lens and subsequently the visual acuity of the patient as the drug dissipates along with the mass and structural material.

An alternative treatment for PCO involves use of laser energy (e.g., from an Nd:YAG laser). Energy from the laser is focused on the posterior lens capsule of the patient to ablate local tissue and cut away the opacified tissue that obstructs the patient's visual field. While effective, this tool uses very high energy and may cause damage to the posterior membrane of the implanted IOL. Alternative variations of PCO treatment include using other lasers such as a femtosecond laser to mechanically cut sections of the capsule. Accordingly, there is a need for IOLs capable of withstanding the rigors of treating PCO.

In various embodiments, the present invention provides a fluid-filled IOL designed to prevent or accommodate damage resulting from laser irradiation or mechanical disturbance due to a procedure used to treat PCO. The IOL may, for example, prevent leakage of fluid through a rupture on the lens surface. In some embodiments, the IOL employs a posterior face that is stronger than the anterior face, e.g., due to material choice, thickness, and/or number of polymeric layers. In other embodiments, the IOL employs multiple interior compartments, and the posterior compartment may include a material that causes self-repair of a tear or other damage that would allow fluid leakage therefrom. In still other embodiments, the posterior compartment is sacrificial; rupture thereof during treatment for PCO is anticipated and does not interfere with optical performance of the lens.

In an aspect, embodiments of the invention feature an intraocular lens that includes, consists essentially of, or consists of a flexible envelope membrane enclosing a liquid and a polymer matrix joined to an interior of the envelope membrane along only a first portion thereof. The polymer matrix contains a pharmaceutical compound therewithin. The envelope membrane is configured to allow diffusion of the pharmaceutical compound through the first portion of the envelope membrane and prevent diffusion of the pharmaceutical compound through a second portion of the envelope membrane different from the first portion.

Embodiments of the invention may include one or more of the following in any of a variety of combinations. The second portion of the envelope membrane may be sealed to prevent diffusion of the pharmaceutical compound therethrough. The pharmaceutical compound may include, consist essentially of, or consist of an anti-inflammatory compound. The polymer matrix may be sandwiched within a membrane bilayer integral with the envelope membrane.

In another aspect, embodiments of the invention feature a method of treating posterior capsule opacification (PCO) in a patient's eye comprising (i) a lens capsule having a posterior surface exhibiting PCO, and (ii) an intraocular lens implanted within the lens capsule. The intraocular lens includes, consists essentially of, or consists of (a) a flexible envelope membrane enclosing a liquid, and (b) a polymer component joined to the envelope membrane along a first portion thereof. The first portion of the envelope membrane faces the posterior surface of the lens capsule. A portion of the posterior surface of the lens capsule is removed. Thereduring, at least part of the first portion of the envelope membrane is removed, thereby forming one or more openings therein. The polymer component substantially prevents passage of the liquid from the envelope membrane through the one or more openings therein.

Embodiments of the invention may include one or more of the following in any of a variety of combinations. The polymer component may be sandwiched within a membrane bilayer integral with the envelope membrane. The polymer component may be joined to an interior surface of the envelope membrane. The polymer component may include, consist essentially of, or consist of a material different from a material of the envelope membrane. The polymer component may include, consist essentially of, or consist of a gel, a hydrogel, phenyl-substituted silicone, and/or parylene. Removing the at least a portion of the posterior surface of the lens capsule may include, consist essentially of, or consist of focusing laser or ultrasound energy thereon. Removing at least part of the first portion of the envelope membrane may include, consist essentially of, or consist of exposure thereof to laser or ultrasound energy.

In yet another aspect, embodiments of the invention feature an intraocular lens that includes, consists essentially of, or consists of a flexible envelope membrane enclosing a liquid, a membrane bilayer integral with the envelope membrane, and an optical property-modifying element sandwiched within the membrane bilayer. The membrane bilayer is disposed along a first portion of the envelope membrane. The optical property-modifying element has at least one optical property different from that of the envelope membrane.

Embodiments of the invention may include one or more of the following in any of a variety of combinations. The optical property-modifying element may include, consist essentially of, or consist of a gel, hydrogel, and/or liquid. A refractive index of the optical property-modifying element may be different from a refractive index of the envelope membrane. A refractive index of the optical property-modifying element may be graded (e.g., increasing or decreasing in a direction toward the center of the intraocular lens or an optical axis thereof). The optical property-modifying element may include a plurality of nanoparticles therewithin. The nanoparticles may include, consist essentially of, or consist of titanium dioxide. A diameter (e.g., an average or maximum diameter) of the nanoparticles may be less than approximately 390 nm.

In another aspect, embodiments of the invention feature a method of implantation of an intraocular lens within a patient's eye having a natural lens disposed within a lens capsule. The intraocular lens includes, consists essentially of, or consists of (i) a flexible envelope membrane enclosing a liquid, and (ii) a polymer component joined to the envelope membrane along a first portion thereof. An opening is formed in the lens capsule. At least a portion of the natural lens is removed (e.g., via the opening in the lens capsule). The intraocular lens is inserted within the lens capsule such that the first portion of the envelope membrane faces the opening in the lens capsule. The polymer component substantially prevents movement or bulging of the envelope membrane through the opening in the lens capsule.

Embodiments of the invention may include one or more of the following in any of a variety of combinations. The polymer component may be sandwiched within a membrane bilayer integral with the envelope membrane. The polymer component may be joined to an interior surface of the envelope membrane. The polymer component may be joined to an exterior surface of the envelope membrane. The polymer component may include, consist essentially of, or consist of a material different from a material of the envelope membrane. The polymer component may include, consist essentially of, or consist of a gel, a hydrogel, phenyl-substituted silicone, and/or parylene. A width (or diameter) of the polymer component may be greater than a width (or diameter) of the opening in the lens capsule. The polymer component may include, consist essentially of, or consist of a material having a higher mechanical strength and/or a lower flexibility than that of a material of the envelope membrane.

In yet another aspect, embodiments of the invention feature an implantable reservoir for containing a liquid. The reservoir includes, consists essentially of, or consists of a biocompatible envelope membrane defining an interior lumen and, joined to the membrane along a portion thereof, a property-modifying element for modifying a physical property of the membrane along the portion.

Embodiments of the invention may include one or more of the following in any of a variety of combinations. The property-modifying element may be joined to an exterior surface of the envelope membrane. The property-modifying element may be joined to an interior surface of the envelope membrane. The property-modifying element may be in the form of a secondary membrane. The secondary membrane may have a mechanical property different from a corresponding property of the envelope membrane. The mechanical property may be material strength, flexibility, shear modulus, toughness, Young's modulus, durometer, and/or ductility. The secondary membrane may have a permeability different from a permeability of the envelope membrane. The secondary membrane may have an optical property different from a corresponding property of the envelope membrane. The optical property may be refraction, transparency, transmission spectrum, absorption spectrum, fluorescence spectrum, and/or color. A refractive index of the property-modifying element may be graded (e.g., increasing or decreasing toward a center of the envelope membrane). The property-modifying element may include, consist essentially of, or consist of a gel. The gel may include, consist essentially of, or consist of a hydrogel and/or a silicone gel. The property-modifying element may be sandwiched within a membrane bilayer integral with the envelope membrane. The property-modifying element may include, consist essentially of, or consist of a plurality of adjacent layers each separated from a neighboring layer by a membrane substantially identical in composition to the envelope membrane. A liquid may be disposed within the lumen. The property-modifying element may be partially or substantially completely soluble in the liquid. The reservoir may be shaped and sized to operate as an intraocular lens. The reservoir may be shaped and sized to operate as a breast implant. At least a portion of the envelope membrane may be permeable to a pharmaceutical. The reservoir may be shaped and sized to operate as a drug-delivery device.

In another aspect, embodiments of the invention feature an implantable reservoir for containing a liquid. The reservoir includes, consists essentially of, or consists of a biocompatible membrane defining an interior lumen and, joined to the membrane along a portion thereof, a polymeric or gel matrix and, embedded therein, a sensor for sensing a property of the reservoir and/or the liquid contained therein. The sensor may include, consist essentially of, or consist of a pressure sensor and/or a flow sensor.

In yet another aspect, embodiments of the invention feature a method of manufacturing an implantable reservoir for containing a liquid. First and second concave membranes are formed. A property-modifying element for modifying a physical property of the membrane is joined to at least one of the membranes along a portion thereof. The first and second concave membranes are joined to form a unitary envelope defining the reservoir.

In another aspect, embodiments of the invention feature an intraocular lens that includes or consists essentially of an envelope membrane defining an interior region, a dividing membrane disposed within the envelope membrane, within the interior region, first and second fluidically separate compartments each defined by an interior surface of the envelope membrane and one of two opposed surfaces of the dividing membrane, a first self-sealing facility (e.g., a self-sealing portion of a membrane or a self-sealing valve) for admitting a filling needle into the first compartment and fluidically sealing upon withdrawal of the filling needle, and a second self-sealing facility (e.g., a self-sealing portion of a membrane or a self-sealing valve) for admitting the filling needle from the first compartment into the second compartment and fluidically sealing upon withdrawal of the filling needle from the second compartment. The second compartment but not the first is fabricated to accommodate damage from exposure to laser energy or mechanical disruption.

Embodiments of the invention may include one or more of the following in any of a variety of combinations. The envelope membrane around the second compartment may be shaped for contact with a posterior portion of a patient's lens capsule (e.g., the lens capsule of a human eye). The envelope membrane around the second compartment may be thicker than (e.g., at least twice as thick as, at least five times as thick as, or even at least 10 times as thick as) the envelope membrane around the first compartment. The envelope membrane around the second compartment may include, consist essentially of, or consist of a plurality of membrane layers. One of the membrane layers may include, consist essentially of, or consist of parylene and another of the membrane layers may include, consist essentially of, or consist of silicone. One or more of the membrane layers may include, consist essentially of, or consist of parylene. One or more of the membrane layers may include, consist essentially of, or consist of silicone. One of the membrane layers may include, consist essentially of, or consist of a first silicone compound and another of the membrane layers may include, consist essentially of, or consist of a second silicone compound having a lower durometer and/or lower modulus than the first silicone compound. The durometer and/or modulus of the second silicone compound may be lower than that of the first silicone compound by at least a factor of two, by at least a factor of five, or even by at least a factor of 10. One or more of the membrane layers may crosslink in response to mechanical damage. At least two of the membrane layers may have different surface energies to prevent leakage. The envelope membrane around the second compartment may have a thickness of at least 10 μm. The envelope membrane around the first compartment may have a thickness no greater than 200 μm, no greater than 5 μm, and/or no greater than approximately one-half of the thickness of the envelope membrane around the second compartment. The envelope membrane around the second compartment may have a thickness of at least 100 μm. The envelope membrane around the first compartment may have a thickness no greater than 50 μm. The envelope membrane around the second compartment may have a thickness of at least 200 μm. The envelope membrane around the first compartment may have a thickness no greater than 100 μm. The envelope membrane around the second compartment may include, consist essentially of, or consist of a material stronger but less elastic than the envelope membrane around the first compartment. The second compartment may be filled with a material promoting self-sealing. The material may be a liquid, powder, and/or gel that cures upon interaction with fluid and/or with the passage of time.

In yet another aspect, embodiments of the invention feature an intraocular lens that includes or consists essentially of an envelope membrane defining an interior region, first and second dividing membranes disposed within the envelope membrane, a first compartment within the interior region, a second compartment within the interior region, an inner compartment within the interior region, a first self-sealing facility (e.g., a self-sealing portion of a membrane or a self-sealing valve), a second self-sealing facility (e.g., a self-sealing portion of a membrane or a self-sealing valve), and a third self-sealing facility (e.g., a self-sealing portion of a membrane or a self-sealing valve). The first compartment is defined by an interior surface of the envelope membrane and a first surface of the first dividing membrane. The second compartment is defined by the interior surface of the envelope membrane and a first surface of the second dividing membrane. The second compartment is fluidically separate from the first compartment. The inner compartment is defined by a second surface of the first dividing membrane and a second surface of the second dividing membrane. The inner compartment is fluidically separate from the first and second compartments. The first self-sealing facility admits a filling needle into the first compartment and fluidically seals upon withdrawal of the filling needle from the first compartment. The second self-sealing facility admits the filling needle into the second compartment and fluidically seals upon withdrawal of the filling needle from the second compartment. The third self-sealing facility admits the filling needle into the second compartment and fluidically seals upon withdrawal of the filling needle from the inner compartment. The second compartment has a volume sufficiently small that rupture thereof does not have a clinically significant effect on optical performance of the lens. Only the second compartment may be filled with air or an inert gas. The first and inner compartments may be partially or substantially filled with one or more liquids.

In another aspect, embodiments of the invention feature an intraocular lens that includes or consists essentially of an envelope membrane defining an interior region and a self-sealing facility for admitting a filling needle into the interior region and fluidically sealing upon withdrawal of the filling needle. The envelope membrane has first and second opposed, expandable faces. The first face includes, consists essentially of, or consists of a first polymeric material and the second face includes, consists essentially of, or consists of a second polymeric material having greater strength and less elasticity than the first polymeric material.

Embodiments of the invention may include one or more of the following in any of a variety of combinations. The second polymeric material may include, consist essentially of, or consist of phenyl-substituted silicone or fluorosilicone. The first polymeric material may include, consist essentially of, or consist of fluorine-free and phenyl-free silicone. The first polymeric material may include, consist essentially of, or consist of fluorine-free and phenyl-free silicone. The second polymeric material may include, consist essentially of, or consist of parylene. The first and second polymeric materials may be the same material. The second face may be thicker than (e.g., at least twice as thick as, at least five times as thick as, or even at least as 10 times as thick as) the first face. The second face may have a thickness of at least 10 μm. The first face may have a thickness no greater than 350 μm, no greater than 5 μm, and/or no greater than approximately one-half of the thickness of the second face. The second face may have a thickness of at least 100 μm. The first face may have a thickness no greater than 50 μm. The second face may have a thickness of at least 200 μm. The first face may have a thickness no greater than 100 μm.

The term “substantially” or “approximately” or “about” means ±10% (e.g., by weight or by volume), and in some embodiments, ±5%. The term “consists essentially of” means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 is a sectional view of a FFIR defined by an envelope and having therein a permeability-changing interior element spanning a portion of the inner surface of the envelope membrane in accordance with embodiments of the invention.

FIG. 2 is a sectional view of a FFIR having therein an interior element containing a diffusible material in accordance with embodiments of the invention.

FIG. 3 is a sectional view of a FFIR having therein an interior element within a sandwich structure in accordance with embodiments of the invention.

FIG. 4 is a sectional view of a FFIR having therein an interior element for locally modifying the mechanical properties of the FFIR in accordance with embodiments of the invention.

FIG. 5 is a sectional view of a FFIR having therein an interior element for locally modifying the optical properties of the FFIR in accordance with embodiments of the invention.

FIGS. 6A-6C are sectional views of breast implants having interior elements in accordance with embodiments of the invention.

FIG. 7 schematically illustrates a basic manufacturing procedure for molding a FFIR in accordance with embodiments of the invention.

FIG. 8 schematically illustrates a manufacturing procedure for molding a FFIR with an exterior element in accordance with embodiments of the invention.

FIG. 9 schematically illustrates a manufacturing procedure for molding a FFIR with an interior element in accordance with embodiments of the invention.

FIG. 10 schematically illustrates a manufacturing procedure for molding a FFIR with an interior sandwich structure in accordance with embodiments of the invention.

FIGS. 11A and 11B are sectional views of FFIRs having interior elements composed of multiple adjacent layers in accordance with embodiments of the invention.

FIGS. 12A and 12B are sectional views of FFIRs having interior elements composed of multiple membrane-separated layers in accordance with embodiments of the invention.

FIG. 13 schematically illustrates various approaches to patterning an interior element of a FFIR in accordance with embodiments of the invention.

FIGS. 14A and 14B illustrate FFIRs with a functional device deployed on the interior and exterior, respectively, in accordance with embodiments of the invention.

FIGS. 15A-15D are sectional views of IOLs with multiple compartments and access to the anterior and posterior sections of the IOL through a valve or membrane in accordance with various embodiments of the invention.

FIGS. 16A-16C are sectional views of IOLs having multiple compartments filled with fluid in accordance with various embodiments of the invention.

FIGS. 17A and 17B are sectional views of IOLs having a posterior region that has been strengthened or selected to withstand PCO treatment in accordance with various embodiments of the invention.

### DETAILED DESCRIPTION

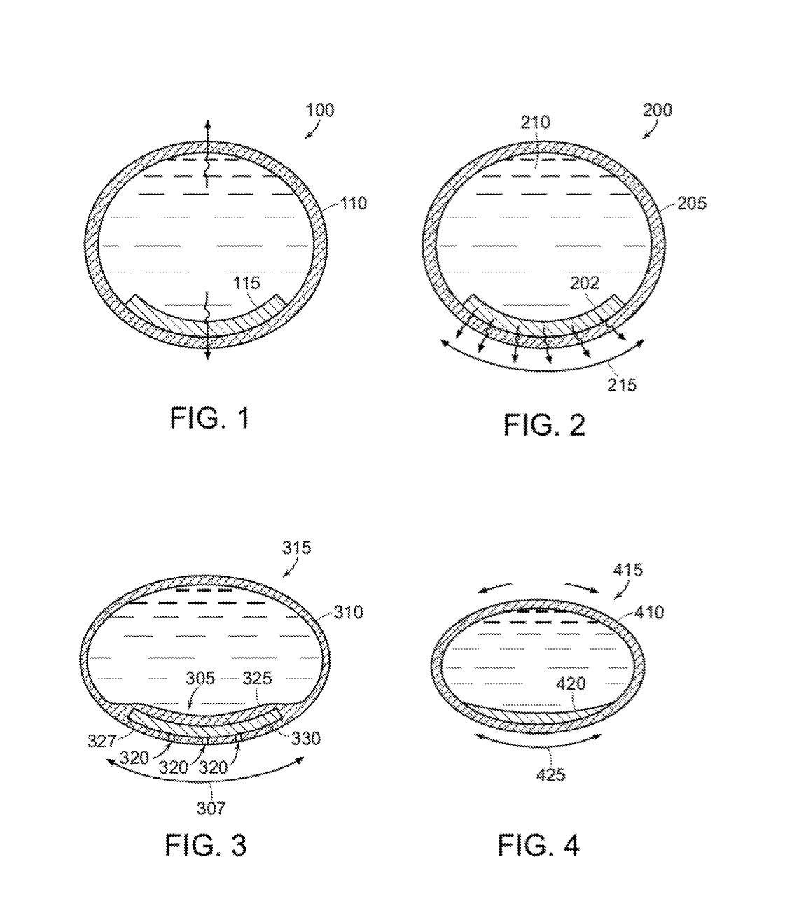

Refer first to FIG. 1, which depicts an approach to modifying the permeability of a FFIR 100. The FFIR 100 is defined by an envelope membrane 110 of substantially uniform composition. For example, the FFIR may be made of a silicone (polydiorganosiloxane, e.g., polydimethylsiloxane or “PDMS”). The envelope membrane 110 may exhibit a permeability P1 to a pharmaceutical or other compound contained within the FFIR 100. For example, it is known that the permeabilities of block copolymers can be varied and optimized for a drug-delivery application by varying the block components. In one well-known system, PDMS/poly (ethylene oxide) (PEO) and PDMS/PEO/poly (methyl methacrylate) (PMMA) block copolymers are utilized, and the permeability of the final block copolymer depends on the PDMS/PEO block size as well as on the PMMA content. Increasing the block size of PDMS, for example, increases the permeability for lipophilic molecules while decreasing the permeability for hydrophilic molecules. The reverse is observed when the block size of PEO is increased. In addition or alternatively, the covalent groups on the PDMS molecule may be altered to change the surface energy of the PDMS, thereby changing uptake and diffusion of molecules through the membrane.

In other embodiments of the invention, the permeability of the FFIR 100 may be altered by various implementations of chemical vapor deposition (CVD) methods. CVD methods of permeability modification provide a tunable and very repeatable manufacturing technique. For example, a first layer of PDMS is fabricated. Next, a second co-polymer (e.g., PDMS with parylene) is chemical vapor deposited within the pores of the first layer of PDMS. The surface of the FFIR is preferably in its natural state or slightly stretched (e.g., taut) during CVD to allow for a uniform deposition to be translatable to the state of the FFIR after implantation. Various characteristics of the CVD technique may be adjusted to obtain the desired permeability to one or more target molecules. Characteristics include but are not limited to the amount of deposition, the percentage cure of the PDMS before deposition, the rate of deposition, and the additional cure after deposition. These characteristics cause the profiles of the two materials and the final composite layer to be different. For example, by depositing at a low rate, the penetration depth may be deeper, causing a less permeable membrane although the material amount is the same. Different second copolymers such as variants of parylene (e.g., parylene-C, parylene-D, parylene-N, parylene-HT, microresist, parylene-C UVF, and parylene with other side chains) may be used depending on the material enclosed in the reservoir and the implantation environment of the FFIR.

A permeability-modifying element 115 is fabricated on or joined to the interior surface of the membrane 110 in order to alter the permeability properties at the region of joinder (i.e., the region where element 115 is joined to the membrane 110). In particular, if the element 115 has a permeability of P2 to the compound of interest and P1>P2 (and particularly when P1>>P2), the local permeability in the region of joinder is will be P2. For example, to confine drug release to the FFIR region outside the region of joinder, the element 115 may have a very low permeability to the drug.

Permeability affects both the rate at which surrounding fluid enters the FFIR 100 as well as the rate at which the internal contents of the FFIR 100 exit. For example, some IOLs are filled with silicone oil. Low membrane permeability to oil is important to prevent leakage into the patient's lens capsule, since the optical properties of the lens are determined by the fill level. For the same reason, low permeability to saline solution is important to prevent entry of body fluid into the lens. In other embodiments, the total net volume is retained and the FFIR benefits from total shape retention by having a first region permeate drug while another region allows fluid in. Alternatively, the inflow of fluid creates an osmotic gradient that alters drug permeation out (directly by fluid, or indirectly by making an intermediate membrane deflect and compress the drug chamber region (e.g., the anterior portion of the IOL to allow for pharmaceutical diffusion to the anterior chamber, the posterior side to allow for pharmaceutical diffusion to the capsule or posterior chamber.) By locally modifying permeability, the same quantity of drug elutes and treats a specific target tissue for longer durations and minimizes systemic side effects to adjacent tissues. This is especially useful for very localized use such as cytoskeletal drugs such as Latrunculin B in the posterior surface of the lens capsule to prevent PCO while preventing unwanted drug loss to the anterior chamber via the flow of aqueous humour. An insert with modified permeability may also be useful for a drug delivery device used inside the eye. Release of a drug into the eye may involve a controllable permeability of the reservoir surface towards the drug molecule. Meanwhile, the permeability of the saline through the reservoir surface should be kept low to avoid the diffusion of the saline from the eye environment into the reservoir by applying a hydrophobic coating on the reservoir internal or external surface. The insert may be modified to meet such particular permeability requirements. The diffusion rate of the pharmaceutical may be modified post-operatively by utilizing PCO or other subsequent laser treatment to target the permeability modifying element 115.

An internal environment of the implant itself may also be altered to modify permeability. The internal environment of the implant (e.g. the drug in the internal reservoir) may be hydrophobic and therefore energetically unfavorable for saline to enter the implant.

FIG. 2 illustrates a FFIR 200 that includes a gel (e.g., a hydrogel, a silicone gel, etc.) or other matrix (e.g., a polymer matrix) 202 as an interior element within an envelope membrane 205. The matrix 202 may be bonded to the interior surface of the membrane 205 or may, in some embodiments, be free-floating. In IOL embodiments, the matrix may be located outside the visual axis of the lens so as not to affect the lens refraction. The matrix 202 contains a pharmaceutical compound that diffuses into a lumen 210 of the FFIR 200 and thereafter through the envelope membrane 205 to the site of implantation. In embodiments in which the matrix 202 is bonded to the interior surface of the membrane 205, the matrix may be fabricated to preferentially release molecules through the side joined to the membrane 205 so that diffusion takes place primarily or solely across the region 215; for example, the unjoined surfaces of the membrane 205 may be sealed to prevent molecular diffusion therethrough.

Sealing to prevent molecular diffusion may be accomplished by incorporating a separate intermediate membrane of low permeability onto the rest of the interior membrane including the interior portion of the matrix 202 to prevent diffusion into the internal volume of the FFIR. Although many methods known in the art may be used, an additional volume of the membrane material (e.g., silicone) may be introduced through one of the refill valves, spin-coated onto the interior surfaces, and cured. Alternatively, a semi-permeable polymer layer that does not alter the optical properties of the FFIR may be applied (e.g., vapor deposition after a mask is placed on the region of the exterior portion of the envelope membrane 205 interfacing with the matrix 202 internally). In one representative implementation, the FFIR is an IOL and the pharmaceutical is an anti-inflammatory compound (e.g., a corticosteroid such as dexamethasone integrated into a porous silicone membrane to diffuse a therapeutic dose, e.g., during the first three days after implantation). A porous silicone envelope membrane 205 may have a thickness between 0.005 mm and 3 mm, or even a thickness between 0.010 mm and 0.500 mm when used as an IOL. For other embodiments in the body, with volumes greater than 300 microliters, the thickness may be, e.g., between 0.020 mm and 3 mm thick. The slow release of the anti-inflammatory compound into the eye helps minimize the inflammation caused by IOL implantation. Alternatively or additionally, an antibiotic (e.g., cephalosporin such as ceftazidime, cefuroxime, or cefazoline, aminoglycosides such as gentamicin, Vancomycin, Amikacin, or other antibiotic) may be embedded in a porous silicone membrane as prophylaxis against endophthalmitis. The material, porosity, permeability, thickness, surface energy, polarity, and number of layers of the envelope membrane 205 may be tailored for each therapeutics' characteristics (molecular structure, concentration, hydrophobicity, and concentration gradient based on half-life, efficacious dose, and clearance rate of target tissue). Incremental volumetric changes of the IOL by the release of drug may change the properties of the IOL including optical properties and flexibility. Such volumetric changes may be minimized by slight overfilling of the IOL to induce a tautness that will offset the volume of drug released. The volumetric change may also be minimized by implementing a collapsible second chamber that preferably and non-reversibly collapses as fluid is transferred to the main chamber via a one-way valve.

FIG. 3 illustrates a sandwich structure 305 that serves as a buffer layer to prevent damage to a region 307 of an envelope membrane 310 of a FFIR 315—damage caused, for example, by surgical manipulations. As noted earlier, treatment of posterior capsule opacification (PCO) (e.g., using a laser such as an Nd:YAG or a femtosecond laser) may damage the posterior membrane of an IOL. The sandwich structure 305 may be located on the posterior side of an IOL in order to improve resistance to this damage.

In PCO treatment, the laser is used to open an aperture in the opacified posterior capsule. In certain cases the IOL itself is unintentionally hit by the surgeon. This error occurs either from improper focusing or from close proximity of the lens to the posterior lens capsule. The damage may include creation of one or more holes 320 in the membrane 310, causing the IOL to leak. In the FFIR 315, the sandwich structure includes interior and outer membrane portions 325, 327 (which are typically, though not necessarily, the same material), and a gel (e.g., a hydrogel) 330 is encapsulated within the layers 325, 327. Notwithstanding unwanted formation of the holes 320, the damaged site remains covered by the gel component 330, which “self-heals” and is not breached. The gel 330 thereby acts as a deformable and flexible plug and prevents leakage. Other examples of the gels that may provide this self-sealing capability include, but are not limited to, silicone gels, polymers, and hydrogels. As a broader definition, the gel is any compound that reacts chemically and/or physically, with or without the presence of a catalyst, by crosslinking, polymerizing or combining to form a more viscous liquid; as used herein, the term “cure” or “curing” refers broadly to the transition of a polymeric liquid to a more viscous state. Curing may occur in a number of ways and at different points in the preparation and implantation process. Many compounds require mixing at least two component parts to initiate cure; others cure over time through changes in molecular conformation. In variations to this embodiment, the gel 330 may be located on the interior surface of the membrane 310 (in the configuration shown in FIG. 2) instead of or in addition to sandwiching between membrane layers.

FIG. 4 illustrates use of an interior element to modify local mechanical properties of the membrane 410 of a FFIR 415. A material 420 (e.g., a gel) different from the material of the membrane 410 is joined to a portion of the interior surface of the membrane. The element 420 has mechanical properties different from those of the membrane 410; for example, the element 420 and the membrane 410 may differ in terms of Young's modulus, tensile strength, and/or other mechanical parameter. Thus, by adding a gel component or a polymer film (e.g., phenyl-substituted silicone or parylene) with higher Young's modulus over a portion 425 of the FFIR surface, the FFIR 415 will be reinforced and, at least across this region, will retain its shape when subjected to external forces. When the FFIR 415 undergoes a tensile stress, for example, the deformation of the region 425 will be smaller than that of the rest of the envelope membrane 410. This is particularly important for applications such as accommodative IOLs, which are constantly loaded in compression or tension by the capsule bag.

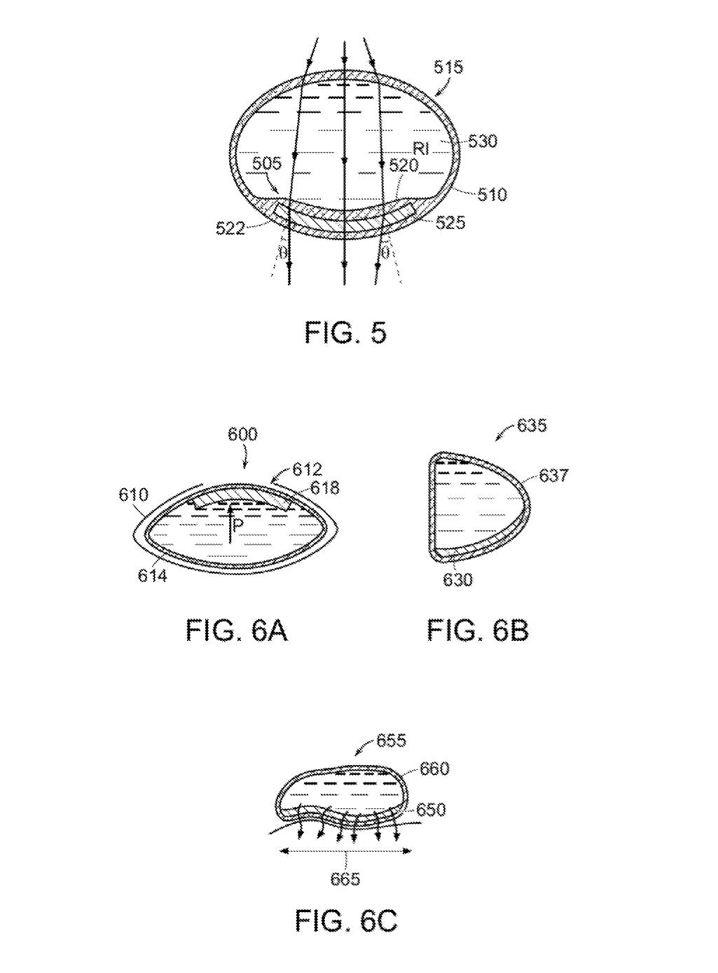

As shown in FIG. 5, a sandwich structure 505 may be used to modify the optical properties of a membrane 510 of a FFIR 515. Here the sandwich structure 505 includes interior and outer membrane portions 520, 522, and a gel (e.g., a hydrogel) or liquid 525 is encapsulated therewithin. The encapsulated material is used to modify the optical properties of the membrane 510, and may have optical properties different from those of the membrane. For example, using a gel material 525 having a higher refractive index than that of the membrane 510 and/or the liquid 530 filling the FFIR 515, the effective refractive index of the FFIR along the sandwich structure 505 is modified. When a light beam passes through the FFIR 515 along the path indicated by the arrows, it may be bent to a greater or lesser extent depending on the material 525. This is important for application such as IOLs, where the refractive index of the FFIR determines the optical power of the lens and how clearly the patient sees after the implantation. The gel material 525 may alternatively or additionally modify other optical properties including but not limited to transmission, absorption, blue blocking, UV blocking, fluorescence, and color thereby adjusting depth of field and light sensitivity.

In addition, the gel 525 may cause the FFIR 515 to act as a gradient-index (GRIN) lens, similar to the natural human lens. The addition of a GRIN component to the FFIR allows the IOL to mimic a natural human lens by having less accommodative potential and a higher index of refraction. Additionally, the GRIN component may be customized to reduce aberration, add astigmatism correction, and act as a toric lens. In other embodiments, the gel itself has a gradient index. For example, a gradient index may be formed by embedding nanoparticles, such as titanium dioxide, into the gel 525 in a non-uniform (e.g., graded) manner. Preferably these nanoparticles have a diameter less than the wavelengths within the visible spectrum (e.g., smaller than approximately 390 nm), and most preferably they are in the Rayleigh scattering range (e.g., of visible light). In addition, the sandwich structure 505 may be used to correct astigmatism, may have a toric shape, and/or may induce or reduce higher-order aberrations in order to increase depth of field. The GRIN lens component may further be altered by using a silicone with refractive index altered by crosslinking capability, adding side chains to the silicone polymer to alter refractive index, use of a multiple layer coating process with different materials, parylene deposition into the PDMS, and/or creating a nanocomposite. This may all be done in a cleanroom, inspected using optical inspection equipment, and then fabricated into the lens.

An interior, property-modifying element as described herein may be used in different types of implantable reservoirs such as IOLs, breast implants, and drug-delivery devices, to name a few. FIG. 6A shows one implementation of the gel component in an IOL 600. The IOL reservoir is implanted in the patient's eye capsule bag 610, which has an opened capsulotomy 612 in the anterior side of the capsule bag 610. Because there is a constant internal pressure inside the IOL 600, the IOL membrane 614 tends to bulge out of the capsulotomy 612 when the IOL 600 has a soft surface. By adding an interior element 618 to the anterior side of the membrane 614, the mechanical strength of the membrane is locally reinforced, discouraging the unwanted bulging without compromising the conformal fit to the capsule bag and translation of accommodation forces by the zonules and ciliary muscles. Thus, in various embodiments of the invention, the width, diameter, or other lateral extent of the interior element 618 is larger than that of the capsulotomy 612, and there is geometric overlap between the interior element 618 and the capsule bag 610.

Similarly, as shown in FIG. 6B, an interior element 630 may benefit a breast implant 635. Once again, an interior element such as a gel may augment the mechanical strength of the breast implant locally, and the envelope membrane 637 is less vulnerable to deformation near the area of the interior element 630.

FIG. 6C illustrates use of a drug-containing interior element 650 in a drug-delivery reservoir 655 along the lines described in connection with FIG. 2. The interior element 650 (e.g., a matrix such as a hydrogel) contains a pharmaceutical compound that diffuses across the membrane 660 where it is joined to the matrix 650 along the region 665. During implantation, the reservoir 655 is oriented so that the region 665 faces the intended site of drug delivery, i.e., the targeted tissue site. The region 665 facing the intended site of drug delivery may conform to a specific curvature or pre-measured shape of an organ (e.g., ocular orbit of an eye to fit and permeate therapeutic to the anterior or posterior chambers of the eye, curvature to wrap at least partially around a blood vessel). Such specific curvature FFIRs may additionally include one or more refill ports to be accessed via post-operative intervention to refill the interior element 618 and fluid within the lumen.

FFIRs in accordance herewith may be manufactured in various ways. The basic FFIR structure may be fabricated conventionally, e.g., using a spin method to form a polymeric (e.g., silicone) balloon suited to use as a medical implant. The interior element may include, consist essentially of, or consist of a thin layer ranging from 5 to 500 μm in thickness; this element, too, may be formed as a layer using a spinning process as discussed below. The interior element may, for example, be made of one or more gel materials, which are initially in the form of a viscous liquid and then cured to a solidified gel. The same approach may be used to form a polymeric interior element.

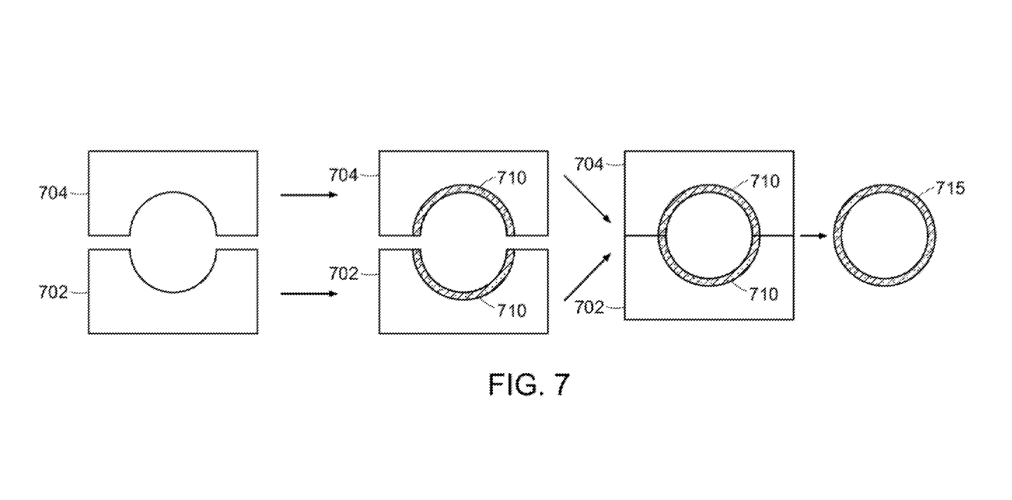

FIG. 7 illustrates a basic manufacturing procedure for molding a FFIR. Two mold halves 702, 704 are used for the molding procedure. First, a layer 710 of the FFIR membrane material, e.g. a viscous silicone liquid, is coated onto the surface of each of the two mold halves 702, 704. Examples of coating techniques include spin coating, molding, compression molding, or spray coating, although various other techniques known by those skilled in the art may be used. The two mold halves 702, 704 are assembled together and the coatings 710 form a complete balloon. The coatings 710 are then cured to form a finished membrane 715, which is removed from the mold halves 702, 704. Examples of curing techniques include exposure to UV or other actinic radiation, baking in elevated temperature, curing at room temperature, curing by exposure to moisture, and various other techniques known to those skilled in the art.

Many of the various manufacturing procedures mentioned above are utilized with FFIR membrane material having a viscosity of at least over 500 centipoise so that the FFIR retains its shape and position by surface tension and molecular interaction with the mold itself during curing. Otherwise, gravity may distort the shape during the curing period. For cure times of over 10 minutes, viscosity of a coated silicone membrane 710 may be over 500 centipoise, over 10,000 centipoise, or even over 15,000 centipose. If spin coating, the membrane material may have a maximum viscosity of 750,000 centipoise for thin coating (although the maximum viscosity is typically limited by the speed of spinning), and the viscosity may be within a range of 10,000-250,000 centipose. Viscosity may be altered by adding a volatile element (e.g., dispersant) to the membrane material (e.g., silicone) which would later evaporate off. The mold may continuously change orientation or be a compression mold during curing to keep a lower viscosity membrane material in the desired shape.

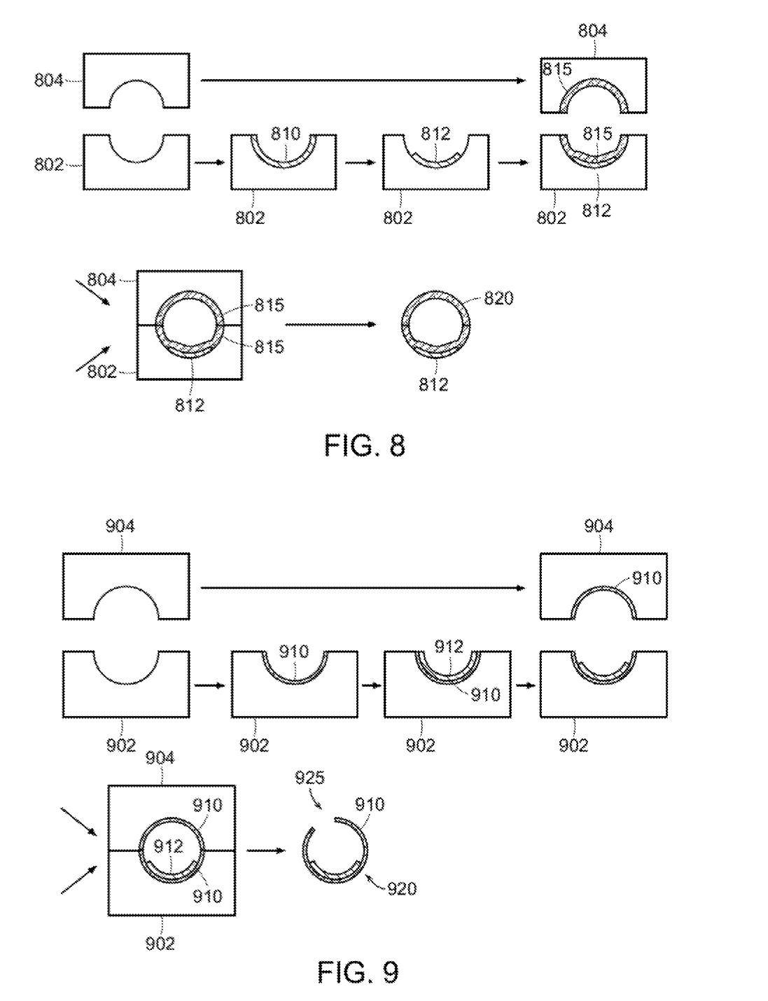

FIG. 8 illustrates an exemplary manufacturing procedure for fabricating a FFIR with an exterior element. First, one of two mold halves 802, 804 is coated with a layer 810 of the material (e.g., a gel material) that will form the exterior element. The desired shape 812 of the gel-covered area is then defined by removing extra gel, which is thereupon cured into a solidified from. After this step, the manufacturing process follows the procedure illustrated in FIG. 7. A layer of the FFIR membrane material 815 is coated onto the surface of each of the two mold halves 802, 804, the FFIR material is cured, and the finished FFIR 820, which includes the exterior element 812 (which has been permanently adhered to the FFIR 820 when the membrane material 815 is cured), is released from the mold halves 802, 804.

FIG. 9 illustrates an exemplary manufacturing procedure to fabricate an FFIR with an interior element. First, one of the two mold halves 902, 904 is coated with a layer 910 of the FFIR membrane material reservoir surface material, e.g., silicone. The coated material is then partially cured, with the result that the membrane material is solidified but remains capable of further cross-linking. Then a layer 912 of the material that will form the interior element (e.g., gel) is coated on top of the membrane material 910. Extraneous material 912 may be removed to define the desired element shape, and the material 912 is cured into solidified form. This and/or a subsequent curing step cross-links the material 912 to the membrane 910.

A layer 910 of the FFIR membrane material is then coated onto the mold half 904. The two mold halves 902, 904 are joined and the coated FFIR membrane material 910 forms a complete balloon. The membrane material is further cured by, for example, exposing to UV radiation or by baking at an elevated temperature. This final curing step permanently adheres the membrane halves together and may strengthen the bond between the membrane material 910 and the interior element 912. Depending on how the finished FFIR 920 is to be used, one or more valves 925 may be incorporated into the FFIR to facilitate fluidic access to the interior thereof for fill, refill, or extraction purposes. Valves may also be incorporated into the FFIR as a variant of a property-modifying element as detailed in U.S. patent application Ser. No. 14/980,116, filed on December 28, 2015, the entire disclosure of which is incorporated by reference herein. Specifically, when valves are integrated into an FFIR used as an IOL, the valve may be out of the central 5 mm of the optical axis and have a thickness no less than 30 μm, no less than 100 μm, or even no less than 250 μm. For an IOL, the maximum thickness of the valve may be limited by surrounding ocular structures, e.g., the iris. Rubbing of the iris may cause ocular inflammation. Therefore, a maximum external thickness of the valve may be about 2 mm, or even about 750 μm. Normal filling may be performed with small-gauge needles, e.g., under 20 ga, or even under 33 ga. A valve for filling the drug chamber may be located at a second location on the FFIR. Such a valve may be constructed of a stress-shielding material, surrounding the valve from the flexible implant. This prevents local deformation of the implant from stretching open the valve itself and releasing the drug. In certain configurations, the valve has a pre-formed slit, preferably accessible by a blunt tip needle for drug replenishment. In others, it is punctured by a sharp needle. However, the latter may be less desirable, as a sharp needle may accidently puncture the wall of the reservoir itself during valve access.

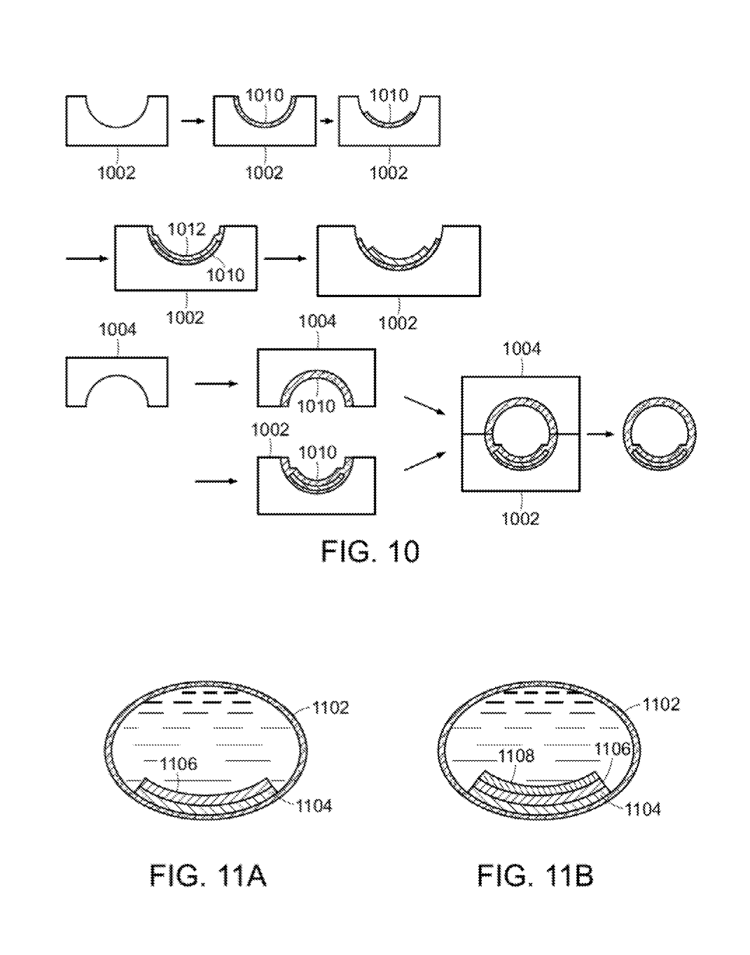

FIG. 10 illustrates manufacture of a FFIR with an internal sandwich structure. First, one of the two mold halves 1002, 1004 is coated with a layer 1010 of FFIR membrane material, e.g., silicone. The coated membrane material is patterned to be slightly larger than the desired sandwich size and is then partially cured, with the result that the membrane material is solidified but remains capable of further cross-linking. A layer of the internal sandwich material 1012 (e.g., gel) is coated on the membrane material 1010. The desired shape of the interior of the sandwich is then defined by removing extra material, following which the layer 1012 is cured into a solidified form. A second layer of the membrane material is coated over the internal material 1012, and a layer 1010 of the membrane material is coated onto the mold half 1002. The two mold halves 1002, 1004 are joined and the coated FFIR membrane material 1010 forms a complete balloon. If desired, the membrane material may be further cured by, for example, exposing to UV radiation or by baking at an elevated temperature.

Although the previous discussion focused largely on a gel interior element, virtually any material that may be coated in liquid or semi-liquid form and then converted into a gel or solid form may be utilized. Such materials include photoresist, wax, hydrogel, silicone (e.g., PDMS, fluorosilicone, phenyl-substituted silicone, etc.), parylene, and various other polymeric materials known to those skilled in the art. Furthermore, suitable materials may be soluble, e.g., materials such as wax, photoresist, sugar or other materials known to those skilled in the art. In such implementations, the internal element may dissolve into the liquid (such as silicone oil) used to fill the FFIR. In still other embodiments, the internal element may be an active component to add functionality to the implantable reservoir, e.g., a pressure sensor, a drug delivery device, or an optical sensor with required electronics and telemetry systems. The active component may be coated with a gel or polymeric material, which may be removed during or after manufacture of the FFIR.

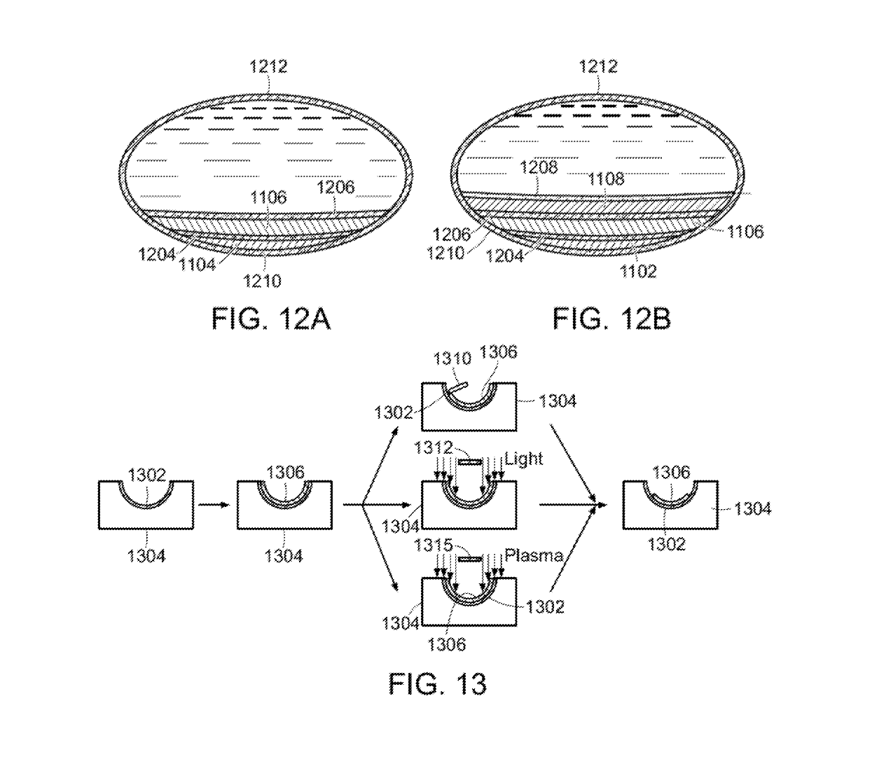

The internal element may be manufactured using a thin-film coating process, e.g., spin coating, dip coating, spray coating, electro-plating, molding (e.g., compression molding), or evaporating from a solvent in a dispersant. In some embodiments, the internal element comprises, consists essentially of, or consists of multiple layers. For example, the internal element within the membrane 1102 may have two adjacent layers 1104, 1106 of gel or other material, as shown in FIG. 11A, or three or more adjacent layers 1104, 1106, 1108 as shown in FIG. 11B. The various layers may be made of the same material or different materials. Moreover, as shown in FIGS. 12A and 12B, two or three layers 1104, 1106, 1108 may be individually encapsulated within the FFIR between separate layers 1204, 1206, 1208 of the membrane material 1210 of the FFIR 1212.

Representative techniques for patterning the interior element are illustrated in FIG. 13. First, optionally, a layer 1302 of the material that will form the FFIR membrane is applied to a mold fixture 1304. A layer 1306 of the material that will form the interior element is then applied to the layer of membrane material (or directly to the mold 1304). In one approach, the excess material of the layer 1306 is removed by using a blade 1310, which mechanically scrapes away unwanted material away along the fixture surface or the underlying layer 1302. In another approach, the material of the layer 1306 is light-sensitive; for example, the material may be a photoresist, which may be patterned using photolithography. A non-transparent mask 1312 selectively shields from exposure the material that is to be retained, and the exposed material is washed away after exposure (using, e.g., conventional photoresist developer). Alternatively, the unwanted material may be etched away using selective exposure to an etchant. A shadow mask 1315 protects the material to be retained from exposure. Etching may be performed using a “wet” or “dry” method. Dry etching techniques include plasma etching, and wet etching techniques involve subjection to a chemical such as KOH or HF. Other suitable approaches include thermal embossing with a patterned mold, lift-off processes, or placing a mask onto the mold surface before coating; after the material 1306 is in place (and before or after cure), the mask is lifted off.

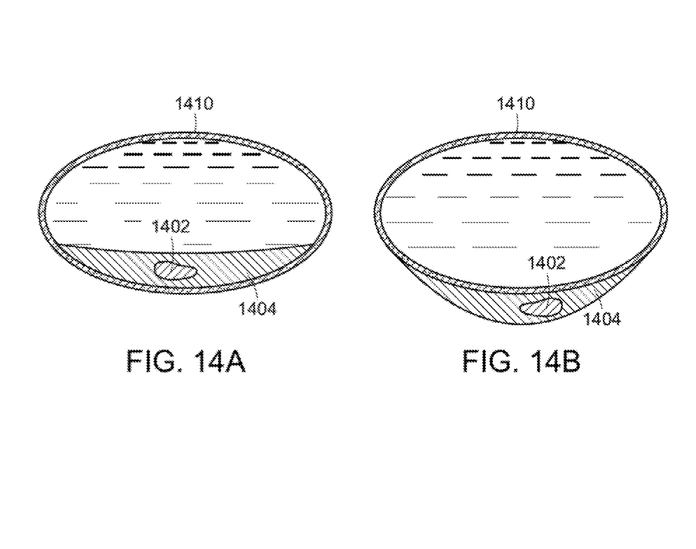

Functional devices may be introduced as illustrated in FIGS. 14A and 14B. In both cases, the functional device (e.g., a sensor such as a pressure sensor or a flow sensor) 1402 is encapsulated in a layer 1404 of gel or polymer, which is applied to the interior or exterior of the FFIR 1410. The encapsulated device 1402 is thereby provided with a soft buffer layer intervening between the device 1402 and the membrane of the FFIR 1410, preserving the flexibility of the FFIR membrane notwithstanding introduction of the device. Moreover, the encapsulating layer 1404 may act as a pressure-transduction layer between the device and the environmental pressure, thereby improving sensing by enlarging the effective region over which the device 1402 responds.

A pressure sensor, for example, may be useful for measuring the internal pressure of the FFIR 1410 to detect damage, but also to monitor the state of the FFIR as it bears on functionality. For example, in an IOL, accommodation of the IOL to change focal length is achieved by shape-changing; a change of shape affects internal pressure, so the pressure sensor may be used in an active feedback loop to adjust the shape of the IOL to provide a target degree of vision correction. Sensors may transmit acquired data using telemetry circuitry, which may be onboard the sensor. For example, the circuitry may be a passive RFID circuit that requires no power or transmission antenna, but instead receives power inductively from a reader and communicates data thereto by varying an electrical property, such as resonant loading, in a temporal pattern.

When the sensor is attached to the outer surface of the reservoir, as shown in FIG. 14B, it may measure the environmental pressure of the FFIR. For example, the sensor may monitor physical parameters such as intraocular pressure, intracranial pressure, and interstitial fluid pressure. Once again, the sensor data may be used in a feedback configuration to adjust the conformation or operation of the FFIR. A combination of both inner and outer sensors and/or multiple sensors may be used to provide multiple sensing modalities and/or provide higher resolution.

In yet another embodiment, the FFIR includes a thin internal dividing membrane and allows for the element to be inserted in a post-implantation process, allowing for the element to be adjustable in size and property. This embodiment allows for an even smaller surgical incision as the FFIR may be folded without the size limitation of a prefabricated element, thereby causing minimal if any optical aberrations to the cornea. Additionally, the internal element may be customized post-implantation to meet the patient's needs (e.g., drug reservoir for drug elution, a further correcting optical element, sensor system, etc.).

In embodiments featuring an internal element configured as a drug reservoir used for drug elution, various components may be modified to provide different drug delivery solutions. In one embodiment, the thin internal dividing membrane is permeable instead of the interfacing envelope membrane. The drug reservoir is refillable and accessible through a separate valve, thereby allowing for a highly concentrated drug solution to be placed in the drug reservoir. The highly concentrated drug (e.g., concentration C1, at 90%) would diffuse through the internal dividing membrane to the larger adjacent lumen where a lower concentration would be maintained (e.g., concentration C2 at 30%). The lower concentration drug in the lumen would diffuse through the permeable regions of the envelope membrane to the target tissue. By maintaining multiple chambers with a separate concentration gradient between each, a highly concentrated drug may be used for space efficiency and stability while delivering the required concentration to be achieved by a combination of permeation rate, permeable membrane surface area, volume of each chamber, and drug clearance rate of the target tissue. The ability to refill with a higher concentration of drug further allows for shorter refill time and minimal change in the FFIR shape between refills which may be critical in FFIRs with shape-defined functionality such as an IOL or shape replacement implant. Such embodiments also solves the problem of fluctuations and sudden drop off in drug delivery that may result as the drug volume depletes in a single-chamber drug elution system. The drug reservoir may have additional coatings (e.g. parylene, silica, etc.) to improve stability.

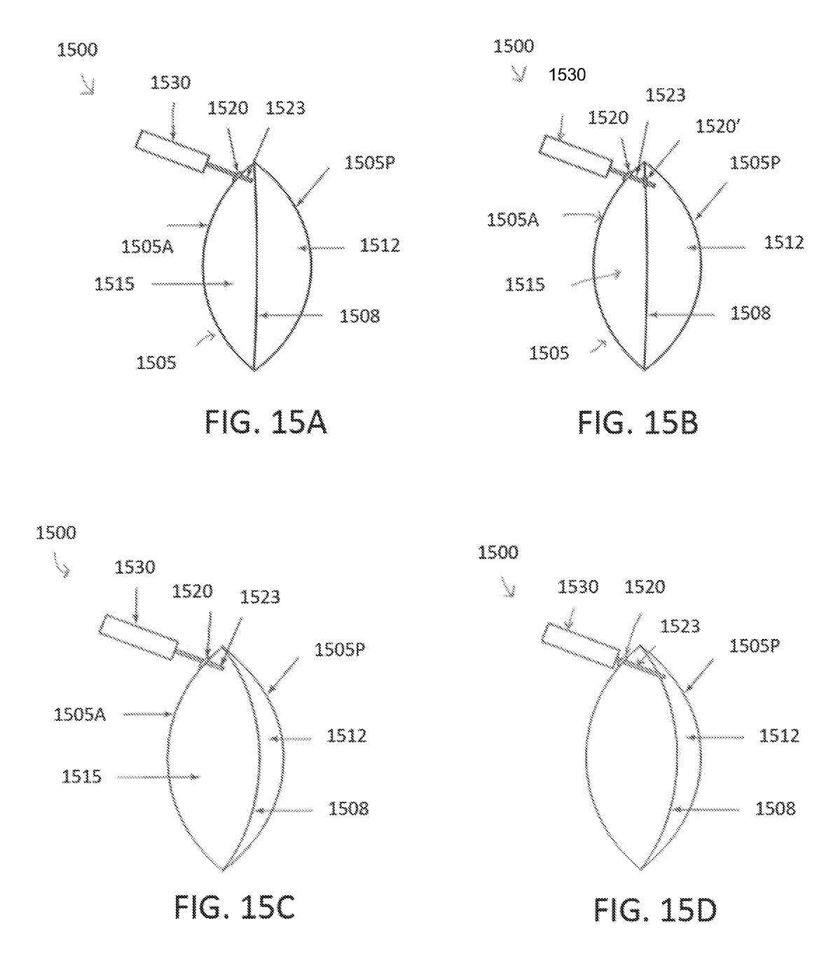

Refer to FIG. 15A, which depicts a cross-sectional view of a fluid-filled IOL 1500 in its nominally filled state, i.e., in which the interior volume of the IOL 1500 is fully filled with liquid without expansion of the exterior membrane. In various embodiments, the fill level may be between 70% and 120% of the nominally filled state in order to adjust the base power of the lens as well as the tautness. Alternatively, the fill level may be based on the individual patient's physical accommodation properties (i.e., ciliary muscle contraction, tension of zonules, and capsule fit).

In this embodiment, the IOL 1500 includes an envelope membrane 1505 defining the interior volume of the IOL and, within the interior volume, a liquid-impermeable dividing membrane 1508. The dividing membrane 1508 divides the interior IOL volume into two fluidically separate compartments 1512, 1515, which may be of roughly equal volume. The portion 1505P of the envelope membrane 1505 surrounding the compartment 1512 may be shaped so that, when filled with liquid, it seats within the posterior portion of a patient's lens capsule. The portion 1505A of the envelope membrane 1505 surrounding the compartment 1515 faces the anterior portion of the patient's lens capsule following implantation. The envelope membrane 1505 may be a unitary structure or may have two separate dome-like segments that are joined (e.g., by heating, adhesive, etc.) along their peripheral edges.

An access port 1520 (e.g., a valve as described above as one type of a property-modifying element) traversing the membrane 1505A admits a fill needle 1523. Because the membrane 1505A faces the anterior capsule following implantation, the port 1520 is readily accessible to the needle 1523 in a clinical setting. The port is self-sealing so that when the needle 1523 is withdrawn, there is no leakage through the port. For example, the port 1520 may include or consist essentially of any of the self-sealing structures, valves, and/or ports described in U.S. patent application Ser. No. 14/980,116, filed on Dec. 28, 2015 (“the '116 application”), the entire disclosure of which is incorporated by reference herein. As shown in FIG. 15B, a second self-sealing needle port 1520′ may be included through the dividing membrane 1508 to admit the needle 1523 into the compartment 1512 after it has passed through the exterior-facing needle port 1520. The port 1520′ may include or consist essentially of any of the self-sealing structures, valves, and/or ports described in the '116 application. Alternatively, the needle 1523 may simply pierce the envelope and/or dividing membrane 1508 to access the compartment 1512, and the membrane(s) self-seal following needle withdrawal. So-called self-healing membrane materials, such as silicone, are well-known in the art.

The IOL 1500 may be stored uninflated inside an insertion tube 1530 of an injector, which includes the needle 1523. During the implantation procedure, the tip of the needle 1523 is inserted into the lens capsule of the patient's eye through a small incision in the cornea. The IOL 1500 is then pushed (e.g., using a plunger in a syringe configuration) out of the insertion tube 1530 and through the needle 1523 into the correct location in the capsule. The IOL 1500 may or may not be stored inside the injector with the needle tip 1523 already inserted into the port 1520. After insertion of the IOL 1500 into the patient's lens capsule, some or all of the IOL 1500 is subsequently filled until the compartments of the IOL 1500 have the desired fill level.

In various embodiments, a low-viscosity, biocompatible polymerizing material with a slow room/body temperature cure is used. Once injected along with any materials of the target internal element (e.g., drug to be eluted, an epoxy of known optical properties, a MEMS sensor), the anterior compartment 1515 fill volume is adjusted to obtain the proper shape of the posterior compartment. Injection of the fill components of the anterior compartment 1515 and the posterior compartment 1512 may also occur simultaneously.

In one implementation, a slow-curing, two-component system such as a customized formulation of a two component polymerizing material commonly used as a biocompatible material, adhesive or sealant for the assembly of medical devices, is injected into the IOL 1500 prior to implantation. The unmixed components may be stored in separate compartments inside the injector or combined just before use. For example, once the IOL 1500 is deployed into the eye, the mixture components travel down a mixing barrel (e.g., within insertion tube 1530) and combine to form the curable liquid, and are thereupon injected into the IOL 1500. The time period required for cure may be long enough to permit re-access to the IOL 1500 to adjust its optical properties; PCO treatment, if necessary, occurs following cure.

The injected fluid may alternatively be cured by exposure to actinic (e.g., ultraviolet) radiation or thermal energy. This on-demand form of activation may be useful during surgery where adjustment of the fluid volume inside the IOL prior to cure is necessary to achieve the desired optical power. The fluid is injected into the IOL in an uncured state and remains uncured until the activation step commences. Activation may be performed before, after or during PCO treatment. For example, a laser, e.g., an Nd:YAG laser, may be used to cure liquid in the posterior compartment 1512 as well as treat the PCO condition. This provides the IOL 1500 with a localized self-sealing mechanism where rupture is likely to occur due to PCO treatment; for example, the unexposed portions of the liquid in the compartment 1512 may remain uncured.

In other embodiments, some compounds (such as hydrogels) require the mixing of a solid and liquid together before setting. As is known in the art, a hydrogel is a network of hydrophilic polymer chains that may take the form of a colloidal gel, in which water is the dispersion medium. Hydrogels are highly absorbent (they can contain over 90% water) natural or synthetic polymeric networks. Hydrogels also possess a degree of flexibility very similar to natural tissue, due to their significant water content.

In one implementation involving the use of hydrogel, the gel polymer powder is introduced into the posterior compartment 1512 of the IOL 1500 prior to implantation (e.g., during manufacture). Once implanted, the IOL 1500 is filled with fluid to the desired fill level. For example, the compartment containing the gel polymer may be filled with fluid osmotically, through a needle that pierces the capsule, or through an access port.

In another embodiment, a crosslinking agent is injected into an uncured or partially cured fluid-filled IOL 1500. Examples of this include hydrogels, acrylic, silicone gels, phenyl-substituted silicones, or fluorosilicones. The crosslinking of the polymer in the presence of the liquid causes molecules of the fluid to be suspended within the matrix of the polymer, trapping the fluid and thereby preventing leakage even with a small rupture of the IOL membrane.

In various embodiments, the volumes of the interior compartments 1512, 1515 may differ. Further, the number of compartments in the interior of the envelope membrane 1505 may vary as well. As shown in FIGS. 15C and 15D, the compartments 1512, 1515 need not be filled to the same level; the relative fill levels may depend on factors such as the different optical properties of the liquids in compartments 1512, 1515, for example. As illustrated, the posterior compartment 1512 may be relatively shallow—indeed, to the point that the cured material forms what is closer to a thickened membrane wall than a separate fluid compartment. In FIG. 15C, the tip of needle 1523 has been inserted into the anterior compartment 1515 of the IOL 1500, which may or may not be filled with the same type of fluid as the posterior compartment 112. FIG. 15D shows the tip of the needle 1523 inserted through the dividing membrane 1508 into the posterior compartment 1512 of the IOL. The fluid injected therein will provide the IOL 1500 with a self-sealing capability. For example, in accordance with various embodiments of the invention, in the event where the chosen PCO treatment (such as using a laser to perform a posterior capsulotomy) were to cause a rupture in the IOL's outer membrane, the fluid contained by the posterior compartment 1512 would not leak through the rupture.

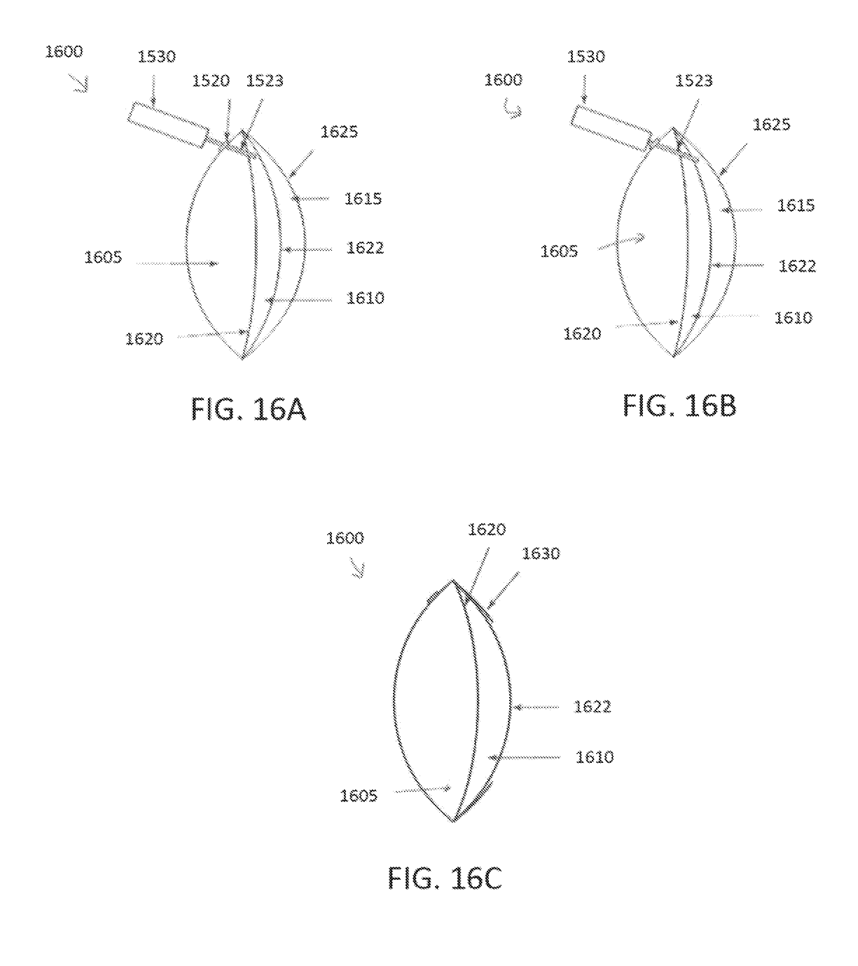

FIGS. 16A-16C depict an IOL 1600 in accordance with various embodiments of the invention. As illustrated, IOL 1600 has three interior compartments 1605, 1610, 1615 defined by a pair of interior impermeable dividing membranes 1620, 1622 within the envelope membrane 1625. The third, posterior compartment 1615 may be sacrificial, i.e., it may have a relatively small volume so that, if the portion of the envelope membrane 1625 surrounding the posterior compartment 1615 is damaged during PCO treatment, leakage of fluid out of the compartment 1615 does not have a clinically significant effect on optical performance of the IOL 1600. At the same time, the presence of fluid within the compartment 1615 during PCO treatment shields the interior membrane 1622 from damage (e.g., a fluid such as a saline or viscoelastic that is naturally absorbed by the eye). In various embodiments of the invention, the fluid in the compartment is index-matched to the surrounding fluid in the eye, e.g. the vitreous or aqueous humour.

Upon deployment of the IOL 1600 from the insertion tube 1530 through the needle 1523 via access port 1520, the needle 1523 is used to fill the anterior compartment 1605 of the IOL via the port 1520 with the desired fluid for that compartment, and then enters the intermediate compartment 1610 through the membrane 1620. As shown in FIG. 16B, the tip of the needle 1523 finally enters the posterior compartment 1615 through the second dividing membrane 1622. The compartments 1605, 1610, 1615 may or may not be filled with the same fluid; for example, different fluids may preferentially be used to create different optical properties or for biocompatible permeation of the fluid into the surrounding tissue. Since the posterior compartment 1615 is designed to be sacrificial in the event of damage, it may be inflated solely with air or an inert gas, which may passively permeate out of the IOL 1600 without damaging the eye or surrounding tissue over a short time period (e.g., less than one week, or even less than 1-3 days). Indeed, the outer compartment may even remain unfilled during the implantation procedure and inflate with air only if a procedure to treat PCO is necessary later during the lifetime of the IOL 1600. Prior to the procedure, the posterior compartment 1615 is accessed (e.g., through a port) and inflated with a liquid (or air).

The result of damage to the envelope membrane 1625 surrounding the posterior compartment 1615 is illustrated in FIG. 16C. Tension in the envelope membrane (which may be optimized by adjusting the fill level of the IOL 1600) has enlarged the rupture therein so as to leave only a relatively small annular region 1630 of the outer membrane that previously surrounded the posterior compartment 1615. This annular region is outside the patient's visual axis and therefore out of view (e.g., outside of 5 mm diameter surrounding the optical axis of the lens), and so will not interfere with lens performance. The posterior compartment has a volume no greater than 30% of the overall interior volume of the lens and equates to at least 50 p.m of depth from the posterior surface and, in various embodiments, 100 to 2000 μm away from the posterior surface when treating PCO. For integration of a drug elution element, the volume may be no greater than 15% of the overall interior volume of the lens.

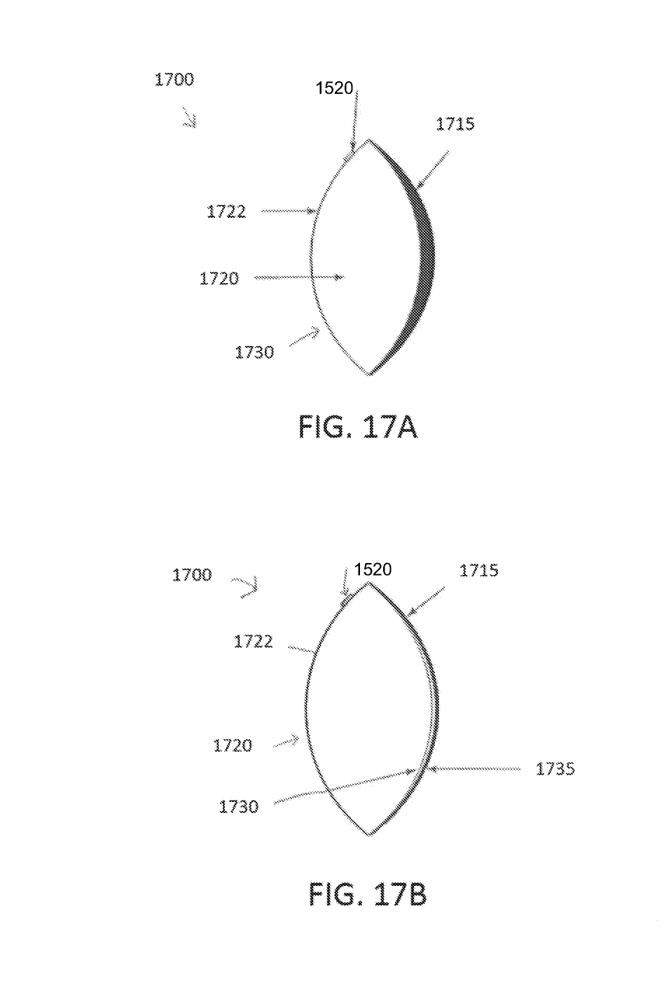

FIGS. 17A and 17B respectively depict IOLs 1700, 1710 in accordance with various embodiments of the present invention. In IOLs 1700, 1710, the posterior side 1715 of the envelope membrane 1720—i.e., the side that seats against the posterior lens capsule—is strengthened or selected to withstand PCO treatment. Envelope membrane 1720 encloses interior compartment 1721 that may be partially or substantially filled with fluid (e.g., liquid). In IOL 1700 depicted in FIG. 17A, the resistance of the posterior side 1715 to rupturing is achieved by material selection and/or by changing the thickness of the material of the side 1715 relative to that of the anterior-facing side 1722. For example, the posterior side 1715 of the membrane 1720 may be made of a strong but optically transparent and biocompatible material that can withstand the chosen PCO treatment. Suitable materials include parylene—i.e., poly (p-xylylene)—a phenyl-substituted silicone, or a fluorosilicone. As understood by those skilled in the art, “silicone” refers to polydiorganosiloxane; the organic substituents along the siloxane chain determine the properties of the silicone polymer. In a phenyl-substituted silicone, not every substituent is a phenyl group; rather, the percentage of phenyl substituents along the chain (with the remainder of the carbon chain atoms bonded to, for example, hydrogen atoms or methyl groups) determines the mechanical properties of the silicone. The same is true for fluorine substitution.

The price of material strength, however, is resistance to deformation; accordingly, a majority of the accommodation offered by the IOL 1700 will arise from the remainder (i.e., the anterior face 1722) of the envelope membrane 1720, which may be, for example, polydimethylsiloxane or other silicone compound whose strength is not enhanced by phenyl or fluorine substitution—and which is therefore more elastic. Alternatively or in addition, the posterior face 1715 may be thicker than the anterior face 1722, making it more resistant to damage induced by PCO treatment but, again, less elastic. For example, the posterior face 1715 may have a thickness of at least 10 μm (and may have a thickness of less than 350 μm or even less than 200 μm), and the anterior face 1722 may have a thickness between 5 μm to 100 μm (or even between 20 μm and 60 μm when using materials with a modulus of elasticity between 0.1 and 8 MPa for the membrane).

FIG. 17B illustrates IOL 1710 in which the posterior portion 1715 of the envelope membrane 1720 is a composite having a plurality of (i.e., two or more) sublayers 1730, 1735 bonded or otherwise joined together in a series of piles. For example, one of the membrane layers 1730, 1735 may be parylene with a thickness between 0.010 μm and 20 μm, and another may be a silicone (e.g., polydimethylsiloxane) with a thickness between 1 μm and 250 μm. Each total bi-layer (e.g., silicone and parylene layer pair) is preferably between 10 μm and 120 μm in thickness. At least two of the membrane layers within the piles may have different surface energies as described above to prevent leakage. Alternatively, one of the membrane layers 1730, 1735 may be a first silicone compound and another membrane layer 1735 is a second silicone compound having a lower durometer and/or lower modulus than the first silicone compound. A low modulus, and low durometer obtained by one of the many characteristics described earlier allows the membrane layers to act as a plug. The softer (e.g., low durometer) material naturally conforms to any hole created in the stiffer material and seals while retaining the original shape of the plurality of membranes. One of the membrane layers may additionally crosslink in response to mechanical or heat damage as part of the PCO treatment, thereby curing. In still another alternative, one of the membrane layers 1730, 1735 may be a phenyl-substituted silicone or fluorosilicone, and another membrane layer 1735 may be a fluorine-free and phenyl-free silicone.

Having described certain embodiments of the invention, it will be apparent to those of ordinary skill in the art that other embodiments incorporating the concepts disclosed herein may be used without departing from the spirit and scope of the invention. For example, various features described with respect to one particular device type and configuration may be implemented in other types of devices and alternative device configurations as well. Accordingly, the described embodiments are to be considered in all respects as only illustrative and not restrictive.

What is claimed is:

1. An implantable reservoir for containing a liquid, the reservoir comprising:
a biocompatible envelope membrane defining an interior lumen; and
joined to the envelope membrane along a portion thereof, a property-modifying element for modifying a physical property of the envelope membrane along the portion.

2. The reservoir of claim 1, wherein the property-modifying element is joined to an exterior surface of the envelope membrane.

3. The reservoir of claim 1, wherein the property-modifying element is joined to an interior surface of the envelope membrane.

4. The reservoir of claim 1, wherein the property-modifying element is in the form of a secondary membrane.

5. The reservoir of claim 4, wherein the secondary membrane has a mechanical property different from a corresponding property of the envelope membrane.

6. The reservoir of claim 5, wherein the mechanical property is at least one of material strength, flexibility, shear modulus, toughness, Young's modulus, durometer, or ductility.

7. The reservoir of claim 4, wherein the secondary membrane has a permeability different from a permeability of the envelope membrane.

8. The reservoir of claim 4, wherein the secondary membrane has an optical property different from a corresponding property of the envelope membrane.

9. The reservoir of claim 8, wherein the optical property is at least one of refraction, transparency, transmission spectrum, absorption spectrum, fluorescence spectrum, or color.

10. The reservoir of claim 8, wherein a refractive index of the property-modifying element is graded.

11. The reservoir of claim 1, wherein the property-modifying element is a gel.

12. The reservoir of claim 11, wherein the gel is a hydrogel.

13. The reservoir of claim 11, wherein the gel is a silicone gel.

14. The reservoir of claim 1, wherein the property-modifying element is sandwiched within a membrane bilayer integral with the envelope membrane.

15. The reservoir of claim 14, wherein the property-modifying element comprises a plurality of adjacent layers each separated from a neighboring layer by a membrane identical in composition to the envelope membrane.

16. The reservoir of claim 1, further comprising a liquid within the lumen, the property-modifying element being soluble in the liquid.

17. The reservoir of claim 1, wherein the reservoir is shaped and sized to operate as an intraocular lens.

18. The reservoir of claim 1, wherein the reservoir is shaped and sized to operate as a breast implant.

19. The reservoir of claim 1, wherein at least a portion of the envelope membrane is permeable to a pharmaceutical, the reservoir being shaped and sized to operate as a drug-delivery device.

20. A method of manufacturing an implantable reservoir for containing a liquid, the method comprising the steps of:
forming first and second concave membranes;
joining to at least one of the membranes, along a portion thereof, a property-modifying element for modifying a physical property of the membrane along the portion thereof; and
joining the first and second concave membranes to form a unitary envelope defining the reservoir.
